(19) United States
(12) Patent Application Publication
(10) Pub. No.: US 2018/0000147 A1
(43) Pub. Date: Jan. 4, 2018

(54) ACTIVITY-ENHANCING SUPPLEMENT

(71) Applicant: Joseph Richardson, Provo, UT (US)

(72) Inventor: Joseph Richardson, Provo, UT (US)

(21) Appl. No.: 15/620,762

(22) Filed: Jun. 12, 2017

Related U.S. Application Data
(60) Provisional application No. 62/348,776, filed on Jun. 10, 2016.

(51) Int. Cl. A23L 33/15; A23L 33/105; A23L 33/17; A61K 31/455; A23L 33/00

(52) U.S. Cl. CPC A23L 33/15; A61K 31/455; A23L 33/40; A23L 33/17; A23L 33/105; A23V 2002/00

(57) ABSTRACT

An activity-enhancing supplement can include niacin and at least two ingredients selected from citrulline, agmatine, an endurance enhancer, a counteracting agent, a nitrogen-containing compound, a muscle building component, branched chain amino acid, and combinations thereof.

### CROSS-REFERENCE TO RELATED APPLICATIONS

This application claims the benefit of U.S. Prov. App. No. 62/348,776, filed on Jun. 10, 2016, which is herein incorporated by reference.

### BACKGROUND

Increasing trends amongst various groups in society toward healthy lifestyles, improved physical appearances, fitness, health awareness, and avoiding preventable diseases, have led to increased physical activity of individuals of these groups. This general increase in physical activity has driven an interest, and a corresponding increase in, the use of nutraceutical supplements. One prominent area of the nutraceutical market includes supplements that enhance mental and physical performance.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

As used herein, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that, which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open ended term in this specification, like “comprising” or “including,” it is understood that direct support should be afforded also to “consisting essentially of” language as well as “consisting of” language as if stated explicitly and vice versa.

As used herein, comparative terms such as “increased,” “decreased,” “better,” “worse,” “higher,” “lower,” “enhanced,” and the like refer to a result achieved from a component or a combination of components, that is measurably different from a result achieved from the component, a substitute component, or another combination of components.

As used herein, “enhanced,” “improved,” “performance-enhanced,” “upgraded,” and the like, when used in connection with the description of a component or a collection of components, refers to a characteristic of the component or the collection of components that provides measurably better results as compared to previously known components or collection of components.

As used herein, the terms “subject” and “individual” can be used interchangeably, and refer to a mammal that may benefit from an activity enhancing supplement. Examples of subjects include humans, and may also include other mammals such as cats, dogs, horses, pigs, cattle, rabbits, and the like. In one specific example, “subject” refers to a human.

As used herein, the term “about” is used to provide flexibility to a numerical range endpoint by providing that a given value may be within 1 from the endpoint. However, it is to be understood that even when the term “about” is used in the present specification in connection with a specific numerical value, that support for the exact numerical value recited apart from the “about” terminology is also provided.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 1.5, 2, 2.3, 3, 3.8, 4, 4.6, 5, and 5.1 individually.

This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

It is common for individuals to use supplements in order to enhance their natural abilities, to improve nutrient levels, increase overall well-being, build a strong immune system, improve focus and concentration, assist in weight loss or weight gain, facilitate sleeping or staying awake, increase energy levels, and the like. In many cases however, supplements that improve energy levels, increase metabolic activity, and fight physical fatigue often comprise high levels of ingredients that can have negative effects on the central nervous system, heart, blood pressure, muscles, and the like. In some examples, these ingredients can be certain stimulants. The effects of many stimulants can vary from individual to individual, including individual tolerance levels to the stimulant, which at times can be adverse. In some instances, stimulates have been known to override the body's normal coping mechanisms for stress, increase wear and tear on the body's tissues, cause a crash in energy levels as the stimulate is depleted from the user's system, and in some cases, can permanently damage the heart. Accordingly, the present disclosure provides activity enhancing supplements that can improve energy levels, stamina, and endurance without causing many of the adverse side effects common in supplements currently on the market.

Generally, an activity-enhancing supplement is disclosed herein, which can be used to increase energy levels and energy production, and increase endurance. In one example, an activity enhancing supplement can include niacin and at least two members selected from the group consisting of agmatine, citrulline, an endurance enhancer, a counteracting agent, a nitrogen-containing compound, a muscle building component, and a branched chain amino acid. One example of a more specific activity enhancing supplement can include at least niacin, agmatine, citrulline, and beta alanine.

Niacin is a tissue targeting component, which in one example can be used as a carrier to transport supplement components into a tissue. In particular, niacin can transport these components to capillaries, where they are taken up by the surrounding tissue. In addition, niacin is a precursor of nicotinamide adenine dinucleotide (NAD), a coenzyme that plays a key role in cellular adenosine triphosphate (ATP) production through the oxidation of nutrients via the citric acid cycle and oxidative phosphorylation. As such, niacin increases ATP production, resulting in increased energy and an increased feeling of wellbeing, and thus can greatly improve the individual's positive association between physical exertion and these positive feelings that result from the use of the formulation. Continued use can also build the body's defenses against conditions such as stroke, increase the efficiency of heart muscle contractions, lower the blood pressure, beneficially affect cholesterol levels, and the like.

In one example, niacin can be present in a supplement formulation at from about 1 wt % to about 10 wt %. In other examples, the niacin can be present at from about 1 wt % to about 5 wt %, at from 1 wt % to 3 wt %, at from about 2.5 wt % to 5 wt %, at from about 3 wt % to about 6 wt %, at from about 2 wt % to about 8 wt %, at from about 3 wt % to about 9 wt %, or at from about 4 wt % to about 8 wt %.

In one example, a supplement formulation can include an amount of niacin that is greater than the recommended daily allowance (RDA), which, for example, would be greater than 16 mg/day for an average adult male. It is noted, that the RDA for niacin, or any other ingredient described herein, can be readily calculated for a given individual by those skilled in the art. In another example, the supplement formulation can include an amount of niacin that is greater than a recommended daily maximum intake of 35 mg/day. In yet other examples, the amounts of niacin in supplement formulations can be at least 5 times, at least 10 times, or at least 15 times greater than the recommended daily maximum intake. In a further example, a supplement formulation can include an amount of niacin that is 5 to 10 times greater than the recommended daily maximum intake. It is noted, that the amounts of niacin and other ingredients described herein can formulated as a single dose, or multiple doses, and can be taken once per day, multiple times per day, prior to physical workout routines, or the like.

In one embodiment, an individual dose can range from 100 mg to 3,000 mg. In another embodiment, an individual dose can range from 250 mg to 3,000 mg. In yet another embodiment, an individual dose can range from 250 mg to 1,500 mg. An individual dose can be taken one or more times per day, and can be tailored for an individual to minimize side effects.

In various formulation examples, the supplement can further comprise various other ingredients, nonlimiting examples of which can include various endurance enhancers, counteracting agents, nitrogen-containing compounds, muscle building components, branched chain amino acids, or the like. These components are active components that can play a role in increasing energy levels and production, increasing stamina and endurance, and building muscle tissues. Nonlimiting examples of muscle building components can include agmatine, citrulline, and the like, including derivatives thereof, such as agmatine sulfate, citrulline malate, and the like.

In one example, a formulation can include niacin, agmatine, citrulline, and β-alanine. The formulation can also include various combinations of other an endurance enhancers, muscle building components, carbohydrates, counteracting agents, branched chain amino acids, nitrogen-containing compounds, and the like. Some examples of individual components that can be combined with niacin are addressed below.

Agmatine is a decarboxylated form of L-arginine, and can be any bioavailable form of agmatine. One nonlimiting example includes agmatine sulfate, which is as follows:

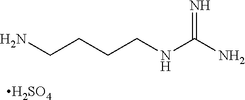

Agmatine sulfate can aid in nutrient partitioning and can increase nitric oxide synthesis. Agmatine sulfate also has physiological actions at imidazoline receptors, and can function as a neurotransmitter and/or a neuromodulator. In one example, agmatine sulfate can block pain. In other examples, agmatine can be metabolized to a carbamoyl phosphate, which in turn can be metabolized into carbamate by the carbamate kinase enzyme that converts adenosine diphosphate (ADP) to ATP.

In one example, a supplement formulation can include agmatine at from about 1 wt % to about 45 wt %. In other examples, supplement formulations can include agmatine at from about 5 wt % to about 15 wt %, at from 1 wt % to about 20 wt %, at from about 5 wt % to about 12.5 wt %, at from about 8 wt % to about 14 wt %, at from about 5 wt % to about 25 wt %, at from about 10 wt % to about 20 wt %, or at from about 15 wt % to about 30 wt %.

Citrulline is an α-amino acid, and can be any bioavailable form of citrulline, including, for example, citrulline malate as follows:

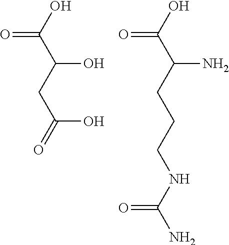

The L-isomer of citrulline can boost nitric oxide levels, which can increase blood flow and reduce fatigue by replenishing depleted nutrients. In one example, a supplement formulation can include citrulline at from about 1 wt % to about 45 wt %. In other examples, supplement formulations can include citrulline at from about 10 wt % to about 20 wt %, at from about 5 wt % to about 20 wt %, at from about 10 wt % to about 25 wt %, at from about 10 wt % to about 30 wt %, at from about 5 wt % to about 25 wt %, at from about 10 wt % to about 30 wt %, or at from about 5 wt % to about 15 wt %.

Agmatine and citrulline are both vasodilators. In one example, the combination of agmatine and citrulline can trap L-Arginine in muscle cells and can increase L-Carnitine levels. Agmatine and citrulline can occupy the same binding sites; however, the inventor has discovered that agmatine and citrulline can be beneficially combined in a single supplement or co-administered without compromising the individual benefits received from the agmatine and citrulline. Supplement formulations containing both agmatine and citrulline can include these ingredients in any beneficial relative amount, and such can be varied depending on the forms of agmatine and citrulline used, and to suit individual needs. Non-limiting examples of ratios of agmatine to citrulline include a 1:1 ratio, a 1:2 ratio, a 1:3 ratio, a 1:4 ratio, a 1:5 ratio, a 1.5:1 ratio, a 2:1 ratio, a 3:1 ratio, and the like. In one example, the agmatine and citrulline can be present at a 1:2 ratio.

In one example, an activity-enhancing supplement can include an endurance enhancer, which can include any useful substance that increases endurance, non-limiting examples of which can include alanine, carnosine, histidine, or the like, including combinations thereof. In one example, the endurance enhancer can be alanine, which can include α-alanine, β-alanine, or the like. In another example, the endurance enhancer can be β-alanine. Beta alanine is a rate limiting precursor of carnosine, and can increase levels of carnosine in the presence of histidine. Carnosine can accumulate in brain and muscle tissue, and can exhibit antioxidant and anti-glycating activities. In one embodiment, β-alanine can be in the upper range of mg/kg for a 100 kg person.

Endurance enhancers can have different effects depending on the nature of the enhancer, the amount of the enhancer, and the user's activity. In some examples, the endurance enhancer can increase muscle hypertrophy and/or assist in fat loss. In other examples, an endurance enhancer can suppress lactic acid formation, thereby decreasing pain and stiffness. Other actions can include neuromodulation through competitive inhibition of taurine, as well as having neural effects similar to the neurotransmitters glycine and gamma-aminobutyric acid (GABA) by binding to GABA_{A} receptors. Other specific effects can include an increase dopamine levels in the nucleus accumbens, as well as anti-anxiety effects.

Endurance enhancer can be included in a supplement formulation at any useful amount. In some examples, a supplement formulation can include an endurance enhancer at from about 1 wt % to about 20 wt % of the supplement. In other examples, supplement formulations can include an endurance enhancer at from about 5 wt % to about 15 wt %, from about 10 wt % to about 20 wt %, from about 5 wt % to about 20 wt %, from about 8 wt % to about 12 wt %, from about 10 wt % to about 14 wt %, or from about 2 wt % to about 16 wt % of the supplement.

In some examples, the activity-enhancing supplement can further comprise a counteracting agent, which can be included in the supplement in order to mitigate depleting effects caused by another component in the supplement. In one example, a counteracting agent can be taurine, which can be added to counteract the taurine depleting effects of β-alanine. In one example, taurine can be included in an equal amount as the endurance enhancer. In other examples, the taurine can be included at a greater or lesser amount than the endurance enhancer. In one embodiment, a β-alanine to taurine ratio can be from 2:1 to 4:1. In a further embodiment, a ratio of β-alanine to taurine in the formulation can be 3:1, 2:1, 1.5:1 or 0.75:1. In another example, the counteracting agent can be aspirin, which is known to reduce the flushing affect associate with high doses of niacin.

The counteracting agent can be included in the supplement at from about 1 wt % to about 20 wt %, from about 5 wt % to about 10 wt %, from about 11 wt % to about 15 wt %, from about 10 wt % to about 20 wt %, or at from about 1 wt % to about 10 wt %.

In a further embodiment, the supplement can comprise a nitrogen-containing compound. As used herein, “nitrogen-containing compound” specifically excludes niacin, as niacin is already present in the formulation. In one example, the nitrogen-containing compound can be nicotinic acid, nicotinamide, NAD, NADH+, and the like, including combinations thereof. In one example the supplement can include nicotinic acid, NAD, and/or NADH+. As a side benefit, these nitrogen-containing compounds can also improve the user's complexion, mood, and positively affect cholesterol levels.

The nitrogen-containing compounds can be included in the supplement at varying amounts. In some examples, the nitrogen-containing compounds can comprise from about 1 wt % to about 20 wt % of the supplement. In other examples, the nitrogen-containing compounds can be present at from about 2 wt % to about 5 wt %, at from about 1 wt % to about 10 wt %, at from about 5 wt % to about 15 wt %, at from about 1 wt % to about 3 wt %, or at from about 12 wt % to about 20 wt %. In some examples, the nitrogen-containing compounds can be included in the formulation at dosage amounts that exceed the maximum daily intake values recommended by the Food and Drug Administration (FDA).

In yet another example, the supplement can further include a muscle building component. In one example, the muscle building component can be a protein, creatine, or a combination thereof. In another example, the muscle building component can be creatine. In one specific non-limiting example, the creatine can be creatine monohydrate. In yet another example, the muscle building component can be a protein, non-limiting examples of which can include whey protein, soy protein, pea protein, animal protein, egg protein, casein protein, and the like, including combinations thereof.

While any amount is contemplated, in some examples the muscle building component can be included in a relatively small amount, such as below industry recommendations, as a cell volumizer, or in a larger amount to improve muscle growth and repair. In one example the muscle building component is creatine, and can be included in a small amount in the supplement to prevent creatine from competing with agmatine for N-methyl-D-aspartic acid (NMDA) receptors. In another example, the creatine can act like a sponge, pulling the necessary building blocks into muscles so these building blocks are readily available for ATP production. Additionally, creatine can speed up the citric acid cycle, which in turn speeds up the production of ATP.

The muscle building component can also be present at a variety of weight ranges in the supplement. In one example, the muscle building component can be present from about 1 wt % to about 20 wt % of the supplement. In other examples, the muscle building component can comprise from about 5 wt % to about 15 wt %, from about 10 wt % to about 20 wt %, from about 5 wt % to about 20 wt %, from about 1 wt % to about 20 wt %, or from about 1 wt % to about 10 wt % of the supplement.

In further examples, the supplement can include a branched chain amino acid, non-limiting examples of which can include leucine, valine, isoleucine, norvaline, 2-aminoisobutric acid, and the like, including combinations thereof. In another example, branched chain amino acids can include L-leucine, L-Valine, and L-Isoleucine. In one example, these branched chain amino acids can be present at a ratio of 2:1:1. In yet another example, this combination of branched chain amino acids can be present at a ratio of 1:1:1. In a further example, this combination of branched chain amino acids are present ratios of about 1:2:1 to 1:2:2, 2:1:3 to 3:1:2, or about 1:3:1 to about 3:3:2. Branched chain amino acids can be included in the activity-enhancing to improve energy levels, aid in recovery from physically rigorous activities, and to increase mental and physical stamina.

Branched chain amino acids can be present at a variety of weight ranges in the supplement. In one example, the branched chain amino acids can be present from about 1 wt % to about 20 wt % of the supplement. In one example, the branched chain amino acids can comprise from about 5 wt % to about 15 wt %, from about 1 wt % to about 7 wt %, from about 1 wt % to about 10 wt %, from about 5 wt % to about 10 wt %, or from about 3 wt % to about 15 wt % of the supplement. It is noted that these weight percentages can be for a single branched chain amino acid moiety, or they can be for the total amount of branched chain amino acid in the formulation, depending on the context.

In yet a further example, the supplement can include caffeine. When included in some examples, caffeine is present at small doses that can be equal to or less than the amount of caffeine found in drinks such as coffee, tea, and soda. Caffeine is a stimulant that affects the central nervous system by blocking the action of adenosine at its receptor, and is known to improve mental alertness. It is believed that caffeine can also increase physical strength, increase endurance, and delay exhaustion in the short-term. When present, caffeine can comprise from about 0.0005 wt % to about 0.003 wt % of the supplement. In another example, the caffeine can comprise from about 0.005 wt % to about 0.002 wt % of the supplement. In another example, the caffeine can comprise 001 wt % of the supplement. In a further example, the caffeine can comprise less than 1 wt % of the formulation.

In still further embodiments, the supplement can include a carbohydrate, non-limiting examples of which can include glucose, maltose, dextrin, fructose 6-phosphate, pyruvate, and the like, including combinations thereof. In one example, the carbohydrate can be a highly branched chain dextrin. In another example, the carbohydrate can be a cyclodextrin. Complex carbohydrates can break down slowly in the body; thereby minimizing the occurrence of insulin spikes and providing a prolonged nutrient source for use in the Krebs cycle. In addition, the inclusion of a highly branched chain dextrin can increase gastric clearing; thereby, reducing the negative effects of some of the components in the formulation on the stomach by allowing for quick clearance through the stomach and into the intestines further permitting a quick absorption of the components. The carbohydrate can also improve weight loss.

Carbohydrates can include a substantial portion of the activity-enhancing supplement. In one example, the carbohydrate can be present from about 1 wt % to about 50 wt % of the supplement. In another example, the carbohydrate can comprise from about 25 wt % to about 30 wt % of the supplement. In other examples, the carbohydrate can comprise from about 30 wt % to about 40 wt %, from about 20 wt % to about 40 wt %, from about 30 wt % to about 50 wt %, or from about 25 wt % to about 35 wt % of the supplement.

The activity-enhancing supplement can further include components added to enhance psychological, physiological, and goal assisting effects. In one example, the components to enhance psychological effects can include noopept, pramiracetam, aniracetam, oxiracetam, choline bitrare, alpha-glycerophosphocholine (alpha-GPC), glycerophosphocholine, choline, tyrosine, or combinations thereof. In one example the component to enhance psychological effects can include tyrosine. Tyrosine is an amino acid that can act as a precursor to norepinephrine. In one example, the component to enhance physiological effects can include L-arginine, electrolytes, D-aspartic acid, or a combination thereof. In one example, the electrolytes can include sodium, potassium, magnesium, or a combination thereof. The component added for goal assisting effects can include L-carnitine, acetyl-1-carnitine, alpha-lipoic acid, 7-oxodehydroepiandrosterone, yohimbe, yohimbine, green tea extract, propionate hunger inhibitors, inulin-propionate ester, probiotics, apple cider vinegar, acidophilus, bifidus, lactobacillus, streptococcus, or a combination thereof. In some examples, the components added to enhance the psychological affects, the physiological affect, and goal assisting affects can be present in the composition from about 1 wt % to about 15 wt %, from about 10 wt % to about 20 wt %, from about 5 wt % to about 10 wt %, or about 1 wt % to about 5 wt %. It is noted that these weight percentages can be for a single component, or they can be for the total amount of these components in the formulation, depending on the context.

It is noted that the various components described herein can be included in a variety of forms or moieties, and any such form or moiety that provide similar physiological benefits are considered to be within the present scope. For example, a given component can be included as the named component, a physiologically active salt or ester, a combination with its various salts or esters, its tautomeric, polymeric and/or isomeric forms, its analog forms, its derivative forms, and the like, including combinations thereof.

The components above are contemplated to be included in the activity supplement in a variety of combinations. In one example, the activity-enhancing supplement can include niacin, agmatine, and citrulline. In another example, the activity-enhancing supplement can include niacin, agmatine, citrulline, and at least one of an endurance enhancer, a counteracting agent, a muscle building component, a nitrogen-containing compound, a branched chain amino acid, caffeine, and a carbohydrate. In a further example, the activity-enhancing supplement can include niacin, agmatine, citrulline, and an endurance enhancer. In yet another example, the activity-enhancing supplement can include niacin, agmatine, citrulline, an endurance enhancer, and a counteracting agent. In another example, the endurance enhancer can be β-alanine and the counteracting agent can be taurine. In yet another example, the activity-enhancing supplement can include niacin, agmatine, citrulline, and a nitrogen-containing compound. In one example, the nitrogen-containing compound can be the combination of nicotinic acid, NAD, and/or NADH.

In another example, the activity-enhancing supplement can include niacin, agmatine, citrulline, an endurance enhancer, and a muscle building component. In one example, the muscle building component can be creatine. In one embodiment, the activity-enhancing supplement can include niacin, along with any combination of agmatine, citrulline, an endurance enhancer, counteracting agent, nitrogen-containing compound, muscle building component, branched chain amino acid, caffeine, and/or a carbohydrate.

In some examples, an activity-enhancing supplement can be formulated in a carrier that is acceptable for use in the nutraceutical, pharmaceutical, nutritional, or food industry arts. Such carriers are well known in the art, and those skilled in the art can readily formulate various formulations using a variety of carriers. The activity-enhancing supplements can be prepared as a single composition or can include several compositions that are packaged together in the form of a kit.

The activity-enhancing supplements presented herein may be provided in a pharmaceutically acceptable vehicle using formulation methods known to those of ordinary skill in the art. The supplement can be provided in any convenient form. When formulated as in an oral dosage form, the supplement can be in the form of a capsule, sachet, tablet, soft gel, lozenges, powder suspension, gel, liquid, beverage, syrup, food, and the like, including appropriate combinations thereof. In one example, the supplement can be in the form of a capsule or tablet. In another example, the supplement can be formulated as a powder that can be mixed with consumable liquids, such as milk, juice, sodas, water, or the like, or formulated as consumable gels or syrups for mixing into other nutritional liquids or foods, or taken alone. The supplement can also be formulated to include pre-measured supplemental foods, such as single serving beverages or bars. In one example, the formulation can be formulated into a nutritional beverage.

In some examples, the supplement can further comprise flavors, coloring agents, spices, nuts, and the like. Flavorings can be in the form of flavored extracts, volatile oils, chocolate flavorings (e.g., non-caffeinated cocoa or chocolate, chocolate substitutes such as carob), peanut butter flavoring, cookie crumbs, crisp rice, vanilla, strawberry, or any commercially available flavoring. Flavorings can be protected with mixed tocopherols. Examples of useful flavorings include but are not limited to pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, cherry oil, walnut oil, cinnamon oil, clove oil, or peppermint oil; peanut butter, chocolate flavoring, vanilla cookie crumb, butterscotch or toffee.

In another example, supplements can further include various preservatives, which can function to extend the shelf life of the supplement. Those skilled in the art are aware of preservatives that can be used in nutritional supplements. Non-limiting examples of preservatives can include potassium sorbate, sodium sorbate, potassium benzoate, sodium benzoate, calcium disodium, EDTA, and the like, including combinations thereof.

The activity-enhancing supplement provided herein can provide various health benefits beyond an increase in energy, stamina, and endurance. These benefits can include decreased sensations of pain and stiffness following physical activity, increased feelings of well-being or decreased feelings of depression, increased sexual drive, improvements in erectile dysfunction, increased weight loss, increased workout intensity, improvement blood cholesterol levels (lower LDL and higher HDL levels), and improvements in skin complexion/less acne. It is also anticipated that the improvements to the user's overall health can decrease the user's chance of experiencing strokes and/or cardiovascular disorders.

Further presented is a method of improving energy, stamina, and endurance levels in an individual by taking an effective amount of the activity-enhancing supplement described herein. In one example, the activity-enhancing supplement can increase energy, stamina, and energy levels in a subject beyond the subject's energy, stamina, and endurance levels in the absence of the activity-enhancing supplement. The activity-enhancing supplement can be administered without cycling off.

The tables below present some example formulation embodiments. These formulation embodiments are merely presented to demonstrate the potential supplement combinations and weight percentages of each component in relation to one another and are not intended to be limiting.

TABLE 1

| Ingredient | Mass (g) | Wt. Percentage |
|---|---|---|
| Niacin | 30 | 10 |
| Citrulline | 135 | 45 |
| Agmatine | 135 | 42.10545 |
| Total | 300 | 100 |

TABLE 2

| Ingredient | Mass (g) | Wt. Percentage |
|---|---|---|
| Niacin | 270 | 10 |
| Citrulline | 1,269 | 47 |
| Agmatine | 621 | 23 |
| β-alanine | 540 | 20 |
| Total | 2700 | 100 |

TABLE 3

| Ingredient | Mass (g) | Weight Percentage |
|---|---|---|
| Creatine | 1 | 15.698 |
| Niacin | 0.12 | 1.883 |
| β-alanine | 1 | 15.698 |
| Citrulline | 1 | 15.698 |
| Agmatine | 0.75 | 11.7739 |
| Taurine | 0.5 | 7.849 |
| Cyclodextrin | 2 | 31.3971 |
| Total | 6.37 | 100 |

TABLE 4

| Ingredient | Mass (g) | Weight Percentage |
|---|---|---|
| Creatine | 1.5 | 12.376 |
| Niacin | 0.37 | 3.052 |
| β-alanine | 1.5 | 12.376 |
| Citrulline | 1.5 | 12.3762 |
| Agmatine | 1.25 | 10.313 |
| Taurine | 1 | 8.250 |
| Cyclodextrin | 5 | 41.254 |
| Total | 12.12 | 100 |

TABLE 5

| Ingredient | Mass | Weight Percentage |
|---|---|---|
| Creatine | 1.3111 g | 14.160 wt % |
| β-alanine | 1.3111 g | 14.160 wt % |
| Citrulline | 1.3111 g | 14.160 wt % |
| Agmatine | 1.000 g | 10.800 wt % |
| Taurine | 0.800 g | 8.640 wt % |
| Niacin | 0.246 g | 2.657 wt % |
| Caffeine | 0.001 g | 0.001 wt % |
| Dextrin | 3.28 g | 35.423 wt % |
| (highly branched & cyclic) | | |

The foregoing detailed description describes the invention with reference to specific exemplary embodiments. However, it will be appreciated that various modifications and changes can be made without departing from the scope of the present invention. The detailed description is to be regarded as merely illustrative, rather than as restrictive, and all such modifications or changes, if any, are intended to fall within the scope of the present invention as described and set forth herein.

### EXAMPLES

### Example 1: Effects of a Stimulate Based Supplement on One Individual Vs. The Effects of the Activity-Enhancing Supplement Described Herein

A forty-year-old, 220 lb., male in good physical condition consumed a control pre-workout supplement commercially available as “Cellucor® C4®” manufactured by Nutrabolt™ (Bryan, Tex.). The ingredients in each dose of Cellucor® C4® are shown in Table 6 below.

TABLE 6
Cellucor C4 Formulation

| Ingredient | Dose |
|---|---|
| Total carbohydrates | 1,000 mg |
| Vitamin C | 250 mg |
| Niacin | 30 mg |
| Vitamin B6 | 0.500 mg |
| Folic Acid | 0.250 mg |
| Vitamin B12 | 0.035 mg |
| Calcium | 20 mg |
| Beta alanine | 1,600 mg |
| Creatine nitrate | 1,000 mg |
| Arginine | 1,000 mg |
| Caffeine Anhydrous | 150 mg |
| N-acetyl-L-tyrosine | 221 mg |
| Velvet Bean seed extract | |
| Tetramethyluric acid | |

Polydextrose, natural and artificial flavors,
citric acid, malic acid, calcium silicate,
silicon dioxide, sucralose, acesulfame
potassium, FD&C Blue Lake #1*
*The amount of these components is not disclosed.

Within a short period of time, the male reported experiencing an increased heart rate (tachycardia), trembling, and anxiety. The male reported that the symptoms were so extreme that the individual did not engage in any physical activity for fear of damaging his heart.

The same male, on a different date, consumed the formulation in Table 5 above. The male did not experience any noticeable symptoms of tachycardia, trembling, or anxiety. However, the male did experience improved energy levels, increased stamina and endurance, and was able to engage in more intense workouts. The male continued to use the formulation in Table 5 on a semi-daily basis and has not reported experiencing any negative long term or short-term side effects.

### Example 2: Study of the Supplements Effect on 15 Adults Ranging from 18-46 Years of Age

Fifteen adults ranging from 18-46 years in age, including both males and females, ingested the formulation in Table 5 before engaging in physical activity over a four-month period. During and following the four month period, the individuals reported a variety of effects including: increased energy levels, increased stamina and endurance, lack of pain following physical exercise, faster recover times, decreased depression, increased sexual drive, improvement in erectile dysfunction, increased caloric consumption, weight loss, increased ability to engage in intense physical activity, a decrease in LDL cholesterol levels, increase in HDL cholesterol levels, clear skin, and improved complexion. The subjects did not report any long term or short term negative side effects.

### Example 3

In one embodiment, an example formulation can include niacin, agmatine and citrulline as an effective “pump” combination, which can cause a nitric oxide boost to blood vessels. Such can result in more efficient mitochondrial processes, with excess NAD and NADH+ being available for ATP production due to a niacin flush effect.

### Example 4

In another embodiment, an example formulation can include niacin, agmatine, citrulline, and beta alanine. This formulation achieves the same results as the formulation of Example 3, but would also assist with endurance through delaying the resistance that naturally occurs due to lactic acid build up in the muscle.

### Example 5

In another embodiment, an example formulation can include niacin, agmatine, citrulline, beta alanine, and creatine. This formulation achieves the same results as the above Example formulations, but also speeds up the production of ATP, and causes muscle cells to become saturated with all of these nutrients through effective cell hydration and volumization.

### Example 6

In another embodiment, an example formulation can include niacin, agmatine, citrulline, beta alanine, creatine, & highly branched chained dextrin. In addition to the results of the above Example formulations, this formulation provides glucose for the production of ATP via the citric acid cycle in mitochondria. Merely including sugar in the formulation, however, has the negative results of causing the pancreas to produce insulin, resulting in severe fatigue. Highly branch chained dextrin is different. It is a complex sugar that is hard to break down. This creates a constant production of glucose to feed the mitochondrial production of ATP without a resulting insulin spike. This is also a beneficial formulation for use by individuals with conditions related to glucose metabolism, such as diabetes and the like.

### Example 7

In another embodiment, an example formulation can include niacin, agmatine, citrulline, beta alanine, creatine, highly branched chain dextrin, and caffeine. Some example formulations may benefit from small amounts of a stimulant, such as caffeine. When used, caffeine should be kept at a low dosage due to its vasodilative properties. As Agmatine and citrulline are also vasodilators, their actions can be impeded by the presence of high levels of caffeine or other stimulants, and can be completely negated if caffeine levels are sufficiently high. Keeping caffeine low thus enhances the effects of agmatine and citrulline. The dosage of caffeine can range from 50 mg to 200 mg.

### Example 8

In another embodiment, an example formulation can include niacin, agmatine, citrulline, beta alanine, creatine, highly branched chain dextrin, and branched chain amino acids and/or taurine. Branched chain amino acids and/or taurine can decrease the effects of pushing the body hard. Taurine specifically because beta alanine is known to deplete taurine, causing a deficiency in the body. It is suggested that you cycle on and off of beta alanine to decrease any negative effects associated with it. Surprisingly enough, taurine supplementation is not recommended with beta alanine in the art. Partly for this reason, taurine levels are kept low in relation to beta alanine so as not to cancel out the effects of beta alanine, yet reduce the need to cycle. The dosage of taurine can be from 50 mg to 1,500 mg.

A dosage of branched chain amino acids can vary depending on the level of exercise. As one example, a typical dosage may be from 500 mg to 3,000 mg of 2:1:1 branched chain amino acids.

What is claimed is:

1. An activity-enhancing supplement, comprising:
niacin;
agmatine;
citrulline; and
β-alanine.

2. The supplement of claim 1, wherein the niacin is present from 1 wt % to 10 wt %.

3. The supplement of claim 1, wherein a weight ratio of the citrulline to the agmatine is greater than 1:1.

4. The supplement of claim 1, wherein the agmatine is agmatine sulfate.

5. The supplement of claim 1, wherein the citrulline is citrulline malate.

6. The supplement of claim 1, wherein a weight percentage of the agmatine is from 1 wt % to 45 wt % and wherein a weight percentage of the citrulline is from about 1 wt % to 45 wt %.

7. The supplement of claim 1, further comprising at least one of carnosine or histidine.

8. The supplement of claim 7, further comprising a green tea extract.

9. The supplement of claim 1, further comprising at least one of protein or creatine.

10. The supplement of claim 9, wherein the creatine is creatine monohydrate.

11. The supplement of claim 9, wherein the protein comprises a member selected from the group consisting of whey protein, soy protein, pea protein, casein protein, animal protein, egg protein, and combinations thereof.

12. The supplement of claim 1, further comprising a carbohydrate selected from the group consisting of glucose, maltose, dextrin, highly branched and cyclic dextrin, fructose 6-phosphate, pyruvate, and combinations thereof.

13. The supplement of claim 12, wherein the carbohydrate is highly branched and cyclic dextrin.

14. The supplement of claim 1, further comprising at least one of taurine or further comprising at least one of taurine or acetylsalicylic acid.

15. The supplement of claim 14, further comprising β-alanine and taurine at a ratio from about 2:1 to 4:1.

16. The supplement of claim 1, further comprising a branched amino acid selected from the group consisting of leucine, valine, isoleucine, norvaline, 2-aminoisobutric acid, and combinations thereof.

17. The supplement of claim 16, wherein the branched chain amino acid comprises L-leucine, L-Valine, and L-Isoleucine in a ratio of 2:1:1.

18. The supplement of claim 1, wherein the supplement excludes caffeine.

19. The supplement of claim 1, wherein the supplement further comprises 1 wt % caffeine or less.

20. An activity-enhancing supplement, comprising:
from 1 wt % to 5 wt % of niacin,
from 10 wt % to 20 wt % of citrulline;
from 5 wt % to 15 wt % of agmatine;
from 10 wt % to 20 wt % of β-alanine
from 10 wt % to 20 wt % of creatine;
from 30 wt % to 40 wt % of dextrin or highly branched and cyclic dextrin;
less than 0.01 wt % caffeine; and
from 5 wt % to 10 wt % taurine.
